(19) United States
(12) Patent Application Publication
(10) Pub. No.: US 2003/0153549 A1
(43) Pub. Date: Aug. 14, 2003

(54) Clavams as protease inhibitors

(76) Inventors: Christopher Joseph Schofield, Oxford (GB); Rupert Charles Wilmouth, Oxford (GB)

Correspondence Address: Quarles & Brady, 411 East Wisconsin Avenue, Milwaukee, WI, 53202-4497, US

(21) Appl. No.: 10/182,072

(22) PCT Filed: Jan. 24, 2001
(86) PCT No.: PCT/GB01/00271

(30) Foreign Application Priority Data
Jan. 26, 2000 (GB) 0001833.3

(51) Int. Cl. A61K 31/5383; A61K 31/424

(52) U.S. Cl. 514/210.07

(57) ABSTRACT

The invention relates to compounds of formula (I) wherein each of X, Y and Z, which may be the same or different, represents hydrogen or an unsubstituted or substituted hydrocarbon radical, typically an alkyl, aryl, including heterocyclic aryl, or non-aromatic heterocyclic radical and Y additionally may represent —COWR wherein W represents O, S or NR^{iv} wherein R^{iv} represents hydrogen or a radical R, and R represents hydrogen or an unsubstituted or substituted hydrocarbon radical for use as protease inhibitors, X, Y and Z being chosen so that they do not react covalently prior to reaction of the β-lactam ring with the target protease.

The present invention relates to clavams.

Soon after the introduction of β-lactam compounds as antibacterials, it became clear that bacteria had evolved resistance mechanisms mediated by β-lactamases which catalyse the hydrolysis of β-lactams to give biologically inactive ring-opened products. Presently the most clinically important β-lactamases employ a catalytic mechanism, involving a nucleophilic serine residue and proceeding via a hydrolytically labile acyl-enzyme intermediate. Clavulanic acid is an important serine β-lactamase inhibitor which itself possesses little antibacterial activity and is consequently administered in combination with an antibiotic.

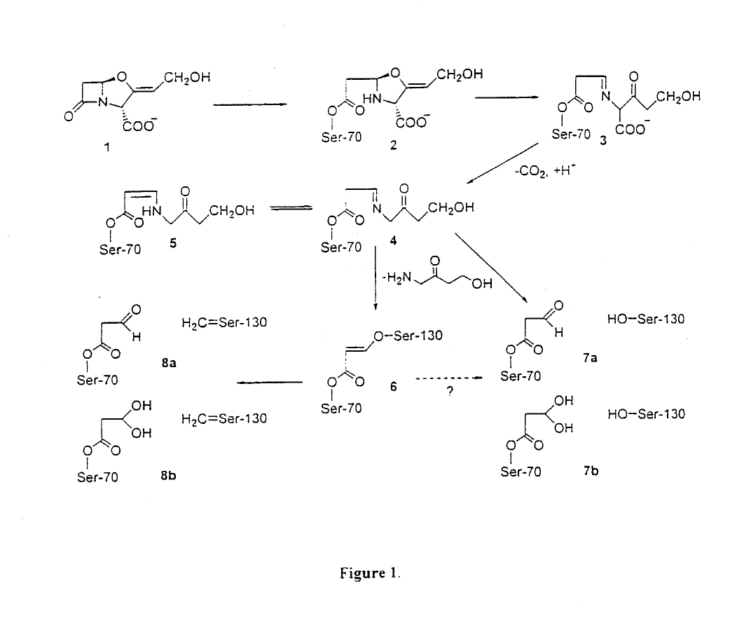

Clavulanic acid (1) inhibits Class A serine β-lactamases via a mechanism involving fragmentation of both its four and five membered rings as shown in FIG. 1 of the accompanying drawings. Spectroscopic and mass spectrometric studies on the clavulanate mediated inhibition of the TEM β-lactamase from Escherichia coli are consistent with a process involving initial acylation and ring-opening of the β-lactams to give 2. Subsequent ring-opening of the five-membered ring is followed by decarboxylation to give the imine/examine acyl-enzyme species 4. Loss of a four carbon fragment can either result in the formation of a relatively stable malonyl semi-aldehyde acyl-enzyme complex 7a which can be reversibly hydrated to give the diol 7b. Alternatively, cross-linking with Ser-130 can occur to form a vinyl ether species 6, the existence of which has been predicted by molecular modelling studies. On prolonged standing hydrolysis of 6 results in elimination from Ser-130 to give a dehydroalanyl residue and the formation of an analogous aldehyde acyl-enzyme complex with Ser-70 (8a). A cryo-crystallographic study on a trapped enzyme-clavulante complex using the Staphylococcus aureus PC 1 β-lactamase proved consistent with the formation of cis-examine attached to Ser-70, as well as the trans-isomer of the decarboxylated examine. The stability of aldehydes 7a and 8a may be due to their preferential reaction to form hydrates (7b, 8b) rather than undergo ester hydrolysis. Displacement of the hydrolytic water, rather than ‘safe reaction’, has also been investigated as a strategy for inhibiting serine β-lactamases. Thus 6α-hydroxyl penicillins have been shown to be inhibitors of TEM β-lactamase.

In recent years β-lactam compounds have also found utility as inhibitors of other ‘serine’ proteases. Elastases have been shown to be inhibited by a variety of mono- and bicyclic β-lactams. It has now been found, according to the present invention, that although clavulanic acid itself possesses little antibacterial activity, as indicated above, certain esters and derivatives of clavulanic acid do in fact act as inhibitors of proteases and, in particular, serine proteases such as elastase.

Accordingly, the present invention provides a pharmaceutical composition which comprises a compound of the formula

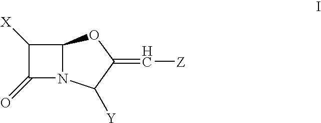

wherein each of X, Y and Z, which may be the same or different, represents hydrogen or an unsubstituted or substituted hydrocarbon radical, typically an alkyl, aryl, including heterocyclic aryl, or non-aromatic heterocyclic radical and Y additionally may represent —COWR wherein W represents O, S or NR^{iv} wherein R^{iv} represents hydrogen or a radical R, and R represents hydrogen or an unsubstituted or substituted hydrocarbon radical together with a pharmaceutically acceptable diluent or carrier.

X, Y and Z should be chosen so that they do not react covalently prior to reaction of the β-lactam ring with the target protease, typically by hydrolysis. In particular if X, Y or Z represent a hydrolysable group such group should be more difficult to hydrolyse than opening of the β-lactam ring. It is preferable that Y, in particular, is not a hydrolysable group. Also it is preferred that X does not represent hydrogen since a substituent in this position enhances acylation and ring opening.

Typical substituents for the radicals X, Y and Z include alkyl (for aryl and heterocyclic groups), aryl or heterocyclic aryl (for alkyl and non-aromatic heterocyclic groups) and non-aromatic heterocyclic (for alkyl, aryl and heterocyclic groups).

The alkyl groups, which can be straight chain or branched, generally have from 1 to 6, especially 1 to 4, carbon atoms, such as methyl; the term “alkyl” includes cycloalkyl which typically has 5, 6 or 7 ring carbon atoms. These groups are optionally substituted by, for example, hydroxyl, alkoxy such as ethoxy, aryloxy e.g. phenoxy such as m-hydroxyphenyloxy, amino including substituted amino such as mono- or di- alkylamino e.g. methylamino, dipropylamino, p-methoxybenzylamino and alkylarylarnino e.g. methyl, 3-pyridylmethyl amino. The aryl groups are typically phenyl groups; they may be substituted by the same groups which can be substituents for the alkyl groups.

Specific examples of X include hydrogen, —C(OH)R₁R₂ or ═CR₁R₂, wherein R₁ and R₂, which may be the same or different, represent hydrogen. alkyl such as methyl, or R₁ and R₂ together complete a 5, 6 or 7 membered carbocyclic ring.

Specific examples of Y include hydrogen, and —COWR as indicated above.

Specific examples of Z include hydrogen, a group of the formula —CHORⁱ where Rⁱ represents hydrogen, alkyl such as methyl, propyl, phenylmethyl and pyridylmethyl e.g. p-methoxy phenylmethyl and 3-pyridyl methyl, or aryl such as phenyl e.g. m-hydroxyphenyl, a group of the formula —CH₂NRⁱRⁱⁱ where Rⁱ and Rⁱⁱ, which may be the same or different, are as defined under Rⁱ, or a group of the formula CH₂(CH₂)ᵣRⁱⁱⁱ where r is 0, 1 or 2 and Rⁱⁱⁱ represents hydrogen or a hydroxy, substituted hydroxy, mercapto, substituted mercapto, azido, cyano, halo, nitro, isothiocyanate, amino, substituted amino aryl, or heterocyclyl group, or the residue of a carbon nucleophile.

The radical R is typically hydrogen or an alkyl, aryl or non-aromatic heterocyclic radical, such as those set out above for X and Z. Suitable radicals R include alkyl, carbonylmethyl, aminoalkyl, alkoxyalkyl, alkanoyloxyalkyl, acylthinoalkyl, haloalkyl, alkenyl, alkynyl, alkanoyl, aralkyl (including heteroaralkyl), aryloxyalkyl, aryl, aralkenyl and aralkoxyalkyl.

The compounds described in the present invention are known or can be obtained from known compounds by methods well known to one of skill in the art. They can generally be prepared from clavulanic acid. Thus compounds in which Y represents CO₂R can be obtained by esterification of the free acid with appropriate modification to provide other substituents at this position.

Substituents X can generally be introduced by reaction of clavulanic acid or the appropriate precursor derivative, protected if required, with an aldehyde or ketone, which gives rise to a hydroxy-containing substituent. This hydroxy- containing substituent can then be reduced or the compound subjected to a dehydration reaction to provide a double-bonded substituent. Further details regarding the introduction of appropriate X substituents can be found by analogy with the processes disclosed in, for example, GB-A-1582884 and Eβ-A-167050, which also disclose typical substituents Z.

Compounds where Z represents an optionally substituted alkyl group can generally be obtained from clavulanic acid or its derivatives using conventional methods, for example as described in GB-A-1508977. An amino group can be introduced by, for example, reaction of an appropriate derivative with sodium azide and subsequent reduction; further details can be found in, for example, U.S. Pat. No. 4,078,068 which includes typical substituents Y.

As indicated, the compounds used in the present invention are found to be effective as inhibitors of proteases and thus the compounds find utility in the treatment of a variety of conditions including cystic fibrosis, thrombosis and arthritis and as cardiovascular therapeutics and anti-viral agents.

The present compounds can be administered in a variety of dosage forms, for example orally such as in the form of tablets, capsules, sugar- or film-coated tablets, liquid solutions or suspensions or parenterally, for example intramuscularly, intravenously or subcutaneously. The present compounds may therefore be given by injection or infusion.

The dosage depends on a variety of factors including the age, weight and condition of the patient and the route of administration. Typically, however, the dosage adopted for each route of administration when a compound of the invention is administered alone to adult humans is 0.001 to 500 mg/kg, most commonly in the range of 0.01 to 100 mg/kg body weight. Such a dosage may be given, for example, from 1 to 5 times daily by bolus infusion, infusion over several hours and/or repeated administration.

A clavam of formula (I) or a pharmaceutically acceptable salt thereof is formulated for use as a pharmaceutical or veterinary composition also comprising a pharmaceutically or veterinarily acceptable carrier or diluent. The compositions are typically prepared following conventional methods and are administered in a pharmaceutically or veterinarily suitable form.

The present compounds may be administered in any conventional form, for instance as follows:

A) Orally, for example, as tablets, coated tablets, dragees, troches, lozenges, aqueous or oily suspensions, liquid solutions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, dextrose, saccharose, cellulose, corn starch, potato starch, calcium phosphate or sodium phosphate; granulating and disintegrating agents, for example, maize starch, alginic acid, alginates or sodium starch glycolate; binding agents, for example starch, gelatin or acacia; lubricating agents, for example silica, magnesium or calcium stearate, stearic acid or talc; effervescing mixtures; dyestuffs, sweeteners, wetting agents such as lecithin, polysorbates or lauryl sulphate. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. Such preparations may be manufactured in a known manner, for example by means of mixing, granulating, tableting, sugar coating or film coating processes.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is present as such, or mixed with water or an oil medium, for example, peanut oil, liquid paraffin, or olive oil.

Aqueous suspensions contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides for example polyoxyethylene sorbitan monooleate.

The said aqueous suspensions may also contain one or more preservatives, for example ethyl or n-propyl p-hydroxybenzoate, one or more colouring agents, and/or one or more sweetening agents such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavouring agents may be added to provide a palatable oral preparation. These compositions may be preserved by the addition of an antioxidant such as ascorbic acid. Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsion may also contain sweetening and flavouring agents. Syrups and elixirs may be formulated with sweetening agents, for example glycerol, sorbitol or sucrose. In particular a syrup for diabetic patients can contain as carriers only products which do not metabolise to glucose or which only metabolise a very small amount to glucose, for example sorbitol.

Such formulations may also contain a demulcent, a preservative and flavouring and colouring agents;

B) Parenterally, either subcutaneously, intravenously, intramuscularly, intrasternally or by infusion techniques, in the form of sterile injectable aqueous or oleaginous suspensions. This suspension may be formulated according to the known art using those suitable dispersing or wetting agents and suspending agents which have been mentioned above. The sterile injectable preparation may also be a sterile injectable solution or suspension in a non-toxic paternally-acceptable diluent or solvent, for example as a solution in 1,3-butane diol.

Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition fatty acids such as oleic acid find use in the preparation of injectables;

C) By inhalation, in the form of aerosols or solutions for nebulizers;

D) Rectally, in the form of suppositories prepared by mixing the drug with a suitable non-irritating excipient which is solid at ordinary temperature but liquid at the rectal temperature and will therefore melt in the rectum to release the drug. Such materials are cocoa butter and polyethylene glycols;

E) Topically, in the form of creams, ointments, jellies, collyriums, solutions or suspensions.

Daily dosages can vary within wide limits and will be adjusted to the individual requirements in each particular case. In general, for administration to adults, an appropriate daily dosage is in the range of about 5 mg to about 500 mg, although the upper limit may be exceeded if expedient. The daily dosage can be administered as a single dosage or in divided dosages.

The following Examples further illustrate the present invention.

Compounds having the following formula were assessed:

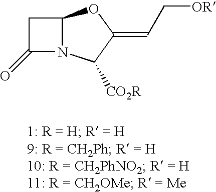

Enzyme assays. Enzyme assays were performed using a Shimadzu 1601PC spectrophotometer equipped with a thermostatted multi-cell transport system. The hydrolysis of the para-nitroanilide substrate (Suc-AlaAlaProAla-pNa, 100 μM) was measured at 405 nm and at a constant temperature of 25° C. in 0.1 M Tris-HCl buffer (pH 7.5). At least quadruplicate measurements of the initial rate were determined at five inhibitor concentrations and data analysed using standard kinetic equations programmed into Excel (Microsoft Corp.) and Grafit (Erithacus Inc.). To aid solubility the clavam inhibitors were dissolved in DMSO to give a final concentration in the assay of 10% (v/v).

NMR experimental procedures. ¹H NMR analyses were performed at 500 MHz on a Bruker AMX500 instrument. Samples (450-500 μl) of PPE (ca. 3 mg) and clavulanic acid derivative (ca 3 mg) were dissolved in 10% (v/v) CD₃CN in D₂O. The temperature was regulated at 303K and the spectra were referenced to internal MeCN at 2.05 ppm.

Electrospray ionisation mass spectrometry. Electrospray ionisation mass spectra were recorded on a Micromass BioQ II-ZS triple quadruple mass spectrometer equipped with an electrospray interface. PPE at 80 pmol μl⁻¹ was incubated with one equivalent of inhibitor. Samples (5 μl) were removed at fixed time points, diluted with water:acetonitrile (1:1 v/v) containing 0.2% (v/v) formic acid to give a protein concentration of 5 pmol μl⁻¹ and analysed immediately. The resulting electrospray mass spectra were calibrated relative to native porcine pancreatic elastase PPE and processed using the MaxEnt algorithm and the spectra reported as centroided data.

As anticipated from prior work on cephalosporins, clavulanic acid (1) itself did not inhibit PPE, but both the benzyl ester (9) and p-nitrobenzyl ester (10) were found to be inhibitors with IC₅₀ values of 184 and 187 μM, respectively, under standard assay conditions. No inhibition was observed with the methoxymethyl derivative (11). It was found that the inhibitory potency of 9 and 10 increased significantly with pre-incubation of the clavam with PPE, indicating that the inhibition was at least partially irreversible. In the case of the benzyl ester derivative (9), 43%, 87% and 93% inhibition was observed after pre-incubation times of 15, 30 and 60 minutes respectively. It is likely that the lack of inhibition observed from clavulanic acid (1) is due to the presence of a negative charge on the carboxylic acid on C-3 which prevents binding within the PPE active site.

¹H NMR (500 MHz) analysis in D₂O/CD₃CN of the incubation of PPE with the benzyl ester derivative (9) demonstrated the production of resonances assigned as arising from clavulanic acid (1) and benzyl alcohol. Their identity was subsequently confirmed by doping experiments. These products were just visible after a one hour incubation period and their concentration was observed to increase after 3 and 12 hours. This indicated that the ester could be hydrolysed by PPE but was unlikely alone to explain the inhibitory activity of 9. The lack of any other detected peaks in the NMR spectrum suggested that any inhibitory complex was not readily hydrolysable. Similar ¹H NMR analysis using the methoxymethyl ester derivative ( 11) showed that hydrolysis of the ester side chain was faster with complete hydrolysis observed after overnight incubation.

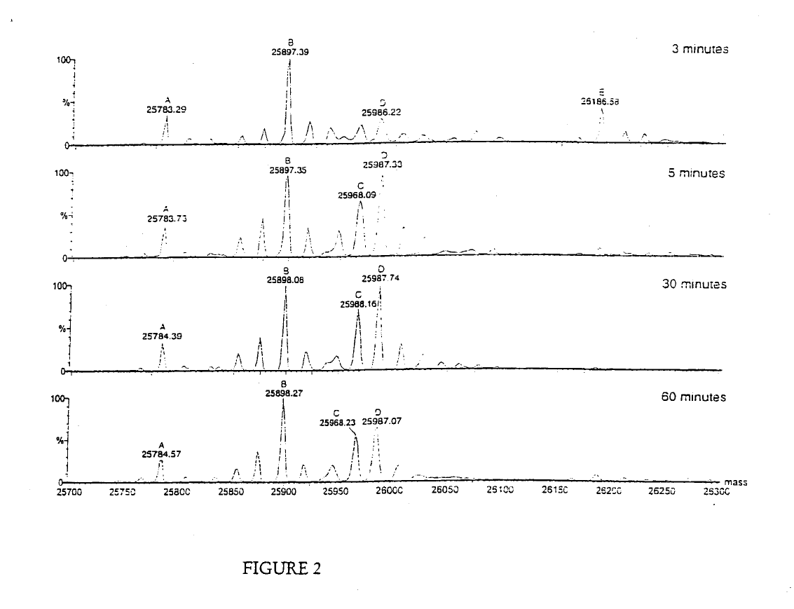

Electrospray ionisation mass spectrometry (ESIMS) analysis of the incubation between the (^{t}Bu)H₃N⁺ salt of clavulanic acid (1) and PPE at time points of between 3 minutes and 5 hours revealed no mass increments relative to native PPE. In contrast, repeats of the ESIMS time course using the benzyl ester derivative (9) (see FIG. 2) demonstrated a peak at 26187 Da after 3 minutes which corresponds to formation of an initial acyl-enzyme complex between 9 and PPE.

Similar results were demonstrated with the p-nitrobenzyl ester (10) except that the formation of the 70 and 88 Da adducts appeared to be slightly faster with significant signals present after 3 minutes. There was no clear evidence for formation of an acyl-enzyme complex formed by ester cleavage, consistent with the proposal that the inhibition of PPE by clavam derivatives occurs via β-lactam cleavage. Presumably, during ester hydrolysis an acyl-enzyme complex is formed only transiently. Somewhat surprisingly, formation of the ‘aldehyde’ adducts was observed with ESIMS analysis of the methoxymethyl ester derivative (11), which was not observed to be an inhibitor under standard assay conditions.

The ca 70 and 88 Da adducts correspond to similar adducts (7a/b) detected in the inhibition of TEM β-lactamase by clavulanic acid (1) and are likely to represent analogous aldehyde and hydrated aldehyde species. After initial acylation of Ser-195 of PPE by the clavam derivatives and subsequent opening of the β-lactams ring, decarboxylation cannot occur due to the presence of the ester functionality on C-3. This explains the absence of any adducts analogous to the acyl-enzyme species 4 seen with TEM β-lactamase inhibition. The ¹H NMR experiments demonstrated that PPE-catalysed hydrolysis of the clavam esters occurs. The lack of inhibition observed for clavulanic acid (1) suggests that this catalytic process competes with inhibition via acylation by the β-lactams ring. Similar hydrolysis of a side-chain ester has also been observed with a series of monocyclic γ-lactams inhibitors of elastase. The rapid PPE-catalysed hydrolysis of the methoxymethyl ester derivative (11) observed by ¹H NMR to give the non-inhibitory clavulanic acid (1) may explain why it was not found to be an inhibitor in the kinetic studies. The rapid reaction (<3 minutes) to form the 70 and 88 Da adducts as observed by ESIMS may indicate that under the unbuffered conditions necessary for the ESIMS analysis, hydrolysis of the methyl ester on C-3 was sufficiently slow for acylation by the β-lactams ring to compete.

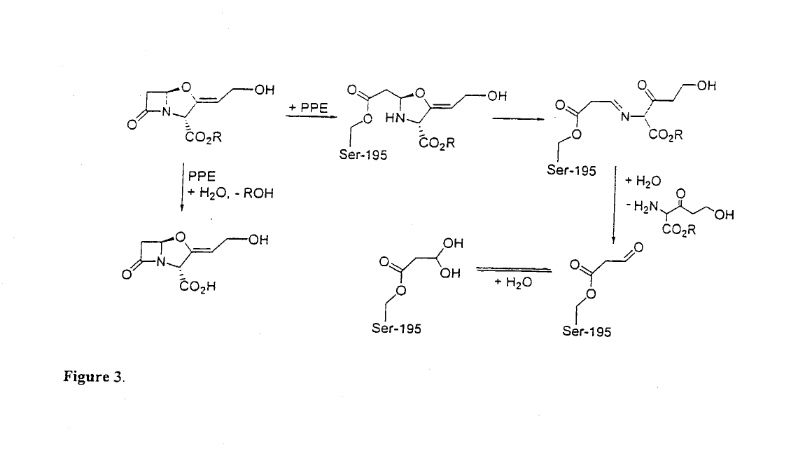

A possible mechanism for the inhibition of PPE by the esterified derivatives of clavulanic acid is shown in FIG. 3. It is assumed that the intermediate imine is hydrolysed by reaction with water molecule within the active site to give rise to the ‘final’ inhibitory aldehyde specified. The imines may be in equilibrium with the isomeric examines (E/Z ratio unknown). PPE also catalyses the hydrolysis of the esters to give clavulanic acid (1) itself.

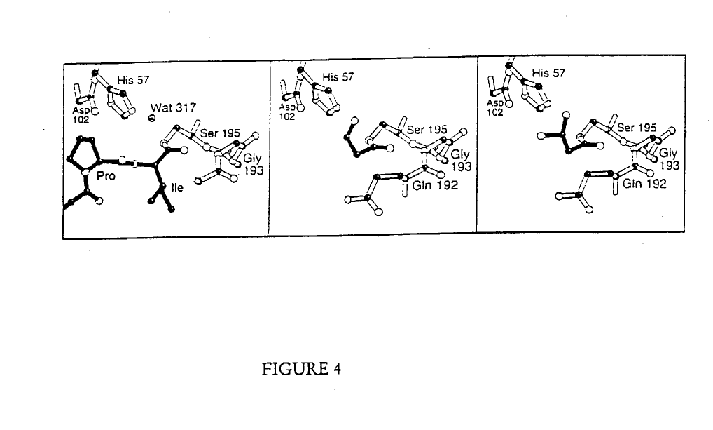

Crystallographic studies on the structure of acyl-enzyme complexes of serine proteases have been reported including one formed between a natural heptapeptide and PPE. This structure reveals a water molecule (Wat-317) positioned above the ester carbonyl, apparently poised for nucleophilic attack, which does not occur due to the pH (ca. 5) of the crystal. Using this structure as a template it seems that a malonyl semi-aldehyde could readily displace or ‘soak up’ the hydrolytic water by reaction with its aldehyde to form a hydrate which would be in a position to form a hydrogen bond with B_{ε2} of His-57. Reference is made to FIG. 4 which shows a model structure of malonyl semi-aldehyde complex (middle) and its hydrated form (right) in PPE. The acyl-enzyme complex between PPE and β-casomorphin-7 is shown for comparison (left). The location of the hydrolytic water (Wat-317) can be seen to be almost coincident with the oxygen of the aldehyde carbonyl. In all cases the inhibitor molecule in shown in black and the enzyme in white. Note that in addition to formation of the aldehyde derivatives, hydration of the intermediate imine may also serve to ‘protect’ the ester linkage.

The fact that analogous aldehydes are formed by reaction of different clavam derivatives with both PPE and TEM β-lactamase, suggests that their formation is more widely applicable to enzyme inhibition. It is possible that the generation of an enzyme-X—COCH₂CR═Y species (X═O, serine proteases; X═S, cysteine proteases; Y═O, S, NR etc.) may be a general way of inhibiting enzymes proceeding via hydrolytically unstable acyl-enzyme complexes. The clavam derivatives of the present invention provide a way of delivering a malonyl semi-aldehyde derivative, which would otherwise be unstable and probably toxic. The generation of other templates capable of delivering the same functionality is a challenge for synthetic chemists.

There are several ways in which the potency of the clavulanic acid (1) derivatives as protease inhibitors may be enhanced. Changing the C-3 ester of Y to a non-hydrolysable functionality removes the second pathway of nucleophilic attack by Ser- 195. Secondly, addition of an alkyl group α to the carbonyl of the β-lactams ring for X has been shown to improve acylation by PPE in a ring of monocyclic β- and γ-lactams inhibitors. When the alkyl group is correctly located in the S₁ subsite of elastase it ensures proper localisation of the β-lactams carbonyl within the oxyanion hole and thus optimises successful nucleophilic attack by Ser-195.

1. A pharmaceutical composition which comprises a compound of the formula 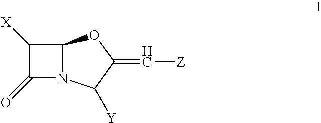
wherein each of X, Y and Z, which may be the same or different, represents hydrogen or an unsubstituted or substituted hydrocarbon radical, typically an alkyl, aryl, including heterocyclic aryl, or non-aromatic heterocyclic radical and Y additionally may represent —COWR wherein W represents O, S or NR^{iv} wherein R^{iv} represents hydrogen or a radical R, and R represents hydrogen or an unsubstituted or substituted hydrocarbon radical, X, Y and Z being chosen so that they do not react covalently prior to reaction of the β-lactam ring with the target protease, together with a pharmaceutically acceptable diluent or carrier.

2. A composition according to claim 1 wherein X is not hydrogen.

3. A composition according to claim 1 or 2 wherein X is an alkyl radical.

4. A composition according to any one of the preceding claims wherein Y is not a hydrolysable group.

5. A compound of formula I as defined in claim 1 for use in the treatment of a condition requiring protease inhibition.

6. Use of a compound of formula I as defined in claim 1 for the manufacture of a medicament for treating a condition requiring protease inhibition.
